(19) United States
(12) Patent Application Publication
(10) Pub. No.: US 2026/0157819 A1
(43) Pub. Date: Jun. 11, 2026

(54) STEREO CAMERA FOR SURGICAL DEVICES AND RELATED SYSTEMS AND METHODS

(71) Applicant: Virtual Incision Corporation, Lincoln, NE (US)

(72) Inventors: Evan Hailey, Lincoln, NE (US); Maven Robert Losey, Lincoln, NE (US); Jay Carlson, Lincoln, NE (US); Parker Durham, Lincoln, NE (US); Shane Farritor, Lincoln, NE (US)

(21) Appl. No.: 19/413,846

(22) Filed: Dec. 9, 2025

Related U.S. Application Data
(60) Provisional application No. 63/729,703, filed on Dec. 9, 2024.

(51) Int. Cl. A61B 90/00; A61B 90/50; H04N 13/194; H04N 13/207; H04N 13/254; H04N 13/302; H04N 23/81

(52) U.S. Cl. CPC A61B 90/37; A61B 90/361; A61B 90/50; H04N 13/194; H04N 13/207; H04N 13/254; H04N 13/302; H04N 23/81; A61B 2090/367; A61B 2090/373; H04N 2213/001

(57) ABSTRACT

Various camera assemblies for robotic surgical systems having a single image sensor and one or more lenses. Further discussed herein is a camera system having a camera assembly, an image signal processor (ISP), and a surgical display. Further discussed herein is a robotic surgical system having a robotic surgical device, a removable camera component having a steerable tip body having a single image sensor and one or more lenses, and a control consol having a surgical display configured to display one or more images captured by the one or more lenses.

### CROSS-REFERENCE TO RELATED APPLICATION(S)

This application claims the benefit under 35 U.S.C. § 119(e) to U.S. Provisional Application 63/729,703, filed Dec. 9, 2024 and entitled “Stereo Camera for Surgical Devices and Related Systems and Methods,” which are hereby incorporated herein by reference in their entireties.

### FIELD

The embodiments disclosed herein relate to various medical devices and related components that can make up a surgical system, including robotic and/or in vivo medical devices and related components. Certain embodiments include various robotic medical devices having camera and display systems incorporated into the devices and system components. Other embodiments relate to various advanced image capturing and display technologies, including stereo or 3D technologies.

### BACKGROUND

Invasive surgical procedures are essential for addressing various medical conditions. When possible, minimally invasive procedures such as laparoscopy are preferred.

However, known minimally invasive technologies such as laparoscopy are limited in scope and complexity due in part to 1) mobility restrictions resulting from using rigid tools inserted through access ports, and 2) limited visual feedback. Known robotic systems such as the da Vinci® Surgical System (available from Intuitive Surgical, Inc., located in Sunnyvale, CA) are also restricted by the access ports, as well as having the additional disadvantages of being very large, very expensive, unavailable in most hospitals, and having limited sensory and mobility capabilities.

Further, known device systems typically require larger device dimensions and thus larger incision sizes in patients than is desirable. For example, standard monocular and stereo cameras that are used in robotic surgical systems—especially, for example, laparoscopic surgical systems—have dimensions that limit size reductions in the overall surgical devices. More specifically, known stereo cameras typically have two separate image sensors positioned side by side, each with a separate lens covering their respective imaging areas. Further, in these known cameras, both image sensors require their own separate support circuitry (to read out the data and provide additional functionality), thereby resulting in the overall size increasing based not only on the number of sensors, but the accompanying separate support circuitry for each sensor.

There is a need in the art for improved cameras for use with various surgical systems, devices, and related methods.

### BRIEF SUMMARY

Discussed herein are various camera assemblies having a single image sensor and one or more lenses. Further discussed herein is a camera system having a camera assembly, an image signal processor (ISP), and a surgical display. Further discussed herein is a robotic surgical system having a robotic surgical device, a removable camera component having a steerable tip body having a single image sensor and one or more lenses, and a control consol having a surgical display configured to display one or more images captured by the one or more lenses.

In Example 1, a camera assembly for a robotic surgical system comprises an elongate camera shaft, a camera body coupled to a proximal end of the elongate camera shaft, a steerable tip disposed at a distal end of the elongate camera shaft, and a flexible section coupled to the elongate camera shaft and the steerable tip body, wherein the steerable tip body is movable in relation to the elongate camera shaft. The steerable tip comprises a single image sensor, one or more lenses disposed distally of the single image sensor, and an illumination component.

Example 2 relates to the camera assembly according to claim 1, wherein the one or more lenses comprise a first lens and a second lens, and wherein the first lens captures a first image and the second lens captures a second image.

Example 3 relates to the camera assembly according to claim 2, wherein the first lens and the second lens form image circles over an entire sensor area of the single image sensor such that the first image and the second image are configured to be captured on the single image sensor.

Example 4 relates to the camera assembly according to claim 2, wherein the first lens and the second lens have a traditional lens design or an anamorphic lens design.

Example 5 relates to the camera assembly according to claim 1, wherein the illumination component is positioned towards an outer edge of a lens housing of the steerable tip body.

Example 6 relates to the camera assembly according to claim 1, wherein the steerable tip body further comprises communication and support components operably coupled to the single image sensor, and a lens housing disposed distally of the single image sensor, wherein the illumination component is disposed within the lens housing and is operably coupled to light fibers extending proximally from the illumination component.

Example 7 relates to the camera assembly according to claim 1, the camera assembly further comprising a communication system configured to transmit visual information from the single image sensor out of the camera assembly, wherein the visual information comprises one or more images captured by the one or more lenses.

Example 8 relates to the camera assembly according to claim 7, wherein the communication system is a serial communication system.

In example 9, a camera system for a robotic surgical system comprises a camera assembly. The camera assembly comprises an elongate camera shaft, a camera body coupled to a proximal end of the elongate camera shaft, and a steerable tip disposed at a distal end of the elongate camera shaft, the steerable tip comprising a steerable tip body comprising a single image sensor, an illumination component, and one or more lenses, the one or more lenses disposed distally of the single image sensor; and a flexible section coupled to the elongate camera shaft and the steerable tip body. The camera system further comprises an image signal processor (ISP) operably coupled to the single image sensor, and a surgical display operably coupled to the ISP, wherein the surgical display is configured to display one or more images captured by the one or more lenses.

Example 10 relates to the camera system according to claim 9, further comprising a communication system configured to transmit visual information from the single image sensor to the ISP, wherein the visual information comprises one or more images captured by the one or more lenses.

Example 11 relates to the camera system according to claim 10, wherein the one or more lenses have a traditional lens design or an anamorphic lens design.

Example 12 relates to the camera system according to claim 12, wherein: the one or more lenses have an anamorphic lens design, the one or more images are distorted one or more images, the communication system is configured to transmit the distorted one or more images to the ISP, and the ISP is configured to correct the distorted one or more images.

Example 13 relates to the camera system according to claim 9, wherein the ISP comprises a black level correction module, a white balance module, a Demosaic module, a color correction module, a digital zoom module, one or more noise reduction modules, an auto exposure module, a sharpening module, and a lens shade correction module.

Example 14 relates to the camera system according to claim 9, further comprising a display console, wherein the display console comprises the ISP and the surgical display.

Example 15 relates to the camera system according to claim 15, wherein the display console comprises a discrete GPU and wherein the discrete GPU is configured to run the ISP.

In Example 16, a robotic surgical system comprises a robotic surgical device comprising an elongate device body comprising a distal end and a proximal end, a removable connection port disposed at the proximal end of the device body, and first and second robotic arms operably coupled to the distal end of the device body. The connection port comprises a device body coupling mechanism disposed within the connection port, a camera receiving opening defined in a proximal end of the connection port, and a camera coupling mechanism disposed within the removable connection port. The robotic surgical system further comprises a removable camera component removably disposable in the camera receiving opening and through the seal package, the removable camera component comprising an elongate camera shaft, a camera body, a flexible section, and a steerable tip having a steerable tip body. The steerable tip body comprises a single image sensor, one or more image lenses disposed distally of the single image sensor, and an illumination component. The robotic surgical system further comprises a control console, the control console comprising a surgical display configured to display one or more images captured by the one or more lenses.

Example 17 relates to the robotic surgical system according to claim 16, wherein the surgical display comprises at least one of a single screen, a plurality of screens, and a pair of achromat doublets.

Example 18 relates to the robotic surgical system according to claim 16, wherein: the one or more lenses comprise a first lens and a second lens; and the surgical display comprises a first display and a second display, the first display corresponding to the first lens and the second display corresponding to the second lens.

Example 19 relates to relates to the robotic surgical system according to claim 16, wherein the control console further comprises hand controllers, and an audio/visual system.

Example 20 relates to the robotic surgical system according to claim 16, wherein the control console further comprises a graphics processing unit (GPU) operatively connected to the single image sensor, the GPU configured to run an image signal processor (ISP) to process visual information from the single image sensor and construct the one or more images to display on the surgical display.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a robotic surgical system, according to one implementation.

FIG. 1B is an alternative external control consol design, according to one implementation.

FIG. 2A is a front view of an autostereoscopic 3D display headset, according to one embodiment.

FIG. 2B is a side view of the headset of FIG. 2A, according to one embodiment.

FIG. 2C is a perspective rear view of the headset of FIG. 2A, according to one embodiment.

FIG. 2D is a perspective front view of the headset of FIG. 2A, according to one embodiment.

FIG. 3 is a front view of the general setup of an autostereoscopic 3D display, according to one embodiment.

FIG. 4 is a console hardware system, according to one implementation.

FIG. 5 is a companion cart, according to one implementation.

FIG. 6 is a companion cart control module, according to one implementation.

FIG. 7 is a robotic device, according to one implementation.

FIGS. 8A and 8B are the robotic device with the camera assembly removed, according to one implementation.

FIGS. 8C and 8D are views of the robotic device with the camera assembly inserted, according to one implementation.

FIGS. 9A and 9B are the camera, according to one implementation.

FIGS. 10A and 10B are the robotic device with a removable nest, according to one implementation.

FIGS. 11A-11D are the tip of the camera assembly, according to one implementation.

FIG. 12 is a standard stereo camera with two side-by-side image sensors, according to one implementation.

FIG. 13 is a stereo camera with a single image sensor, according to one implementation.

FIG. 14 is a top down view of the tip housing with a dual lens-stack architecture, according to one implementation.

FIG. 15 is a standard lens stack design for a single image sensor, according to one implementation.

FIG. 16 is a resulting image from a standard lens stack design, according to one implementation.

FIG. 17 is an anamorphic lens stack design for a single image sensor, according to one implementation.

FIG. 18 is a resulting image from an anamorphic lens stack design before and after interpolation, according to one implementation.

FIGS. 19A-19D are another embodiment of a camera imager, according to one implementation.

FIG. 20 is a traditional video pipeline, according to one implementation.

FIG. 21 is an alternative video pipeline, according to one implementation.

FIG. 22 is a software-based Image Signal Processor system, according to one implementation.

FIG. 23 is an overall system, according to one implementation.

### DETAILED DESCRIPTION

The various systems and devices disclosed herein relate to devices for use in medical procedures and systems. More specifically, various embodiments relate to various medical devices, including robotic devices and related methods and systems, and more specifically to the camera or image capturing devices incorporated into any of those devices and systems.

It is understood that the various embodiments of cameras disclosed herein can be incorporated into or used with not only the specific robotic devices and systems disclosed or contemplated herein, but can also be incorporated into or used with any other known medical devices, systems, and methods that utilize one or more cameras. For example, the various camera and/or imaging sensor embodiments disclosed herein may be incorporated into or used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. patent application Ser. No. 15/966,606 (filed on Apr. 30, 2018 and entitled “Methods, Systems, and Devices for Surgical Access and Procedures”), U.S. patent application Ser. No. 12/192,663 (filed on Aug. 15, 2008 and entitled “Medical Inflation, Attachment, and Delivery Devices and Related Methods”), U.S. patent application Ser. No. 15/018,530 (filed on Feb. 8, 2016 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. patent application Ser. No. 15/888,723 (filed on Feb. 5, 2018 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”) , U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. patent application Ser. No. 16/512,510 (filed on Jul. 16, 2019 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. patent application Ser. No. 16/123,619 (filed on Sep. 6, 2018 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. patent application Ser. No. 15/661,147 (filed on Jul. 27, 2017 and entitled “Robotic Devices with On Board Control & Related Systems & Devices”), U.S. patent application Ser. No. 13/833,605 (filed on Mar. 15, 2013 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. patent application Ser. No. 16/293,135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. patent application Ser. No. 15/687,787 (filed on Aug. 28, 2017 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. patent application Ser. No. 15/894,489 (filed on Feb. 12, 2018 and entitled “Methods, Systems, and Devices Relating to Force Control Surgical Systems”), U.S. patent application Ser. No. 14/212,686 (filed on Mar. 14, 2014 and entitled “Robotic Surgical Devices, Systems, and Related Methods”), U.S. patent application Ser. No. 14/334,383 (filed on Jul. 17, 2014 and entitled “Robotic Surgical Devices, Systems, and Related Methods”), U.S. patent application Ser. No. 14/853,477 (filed on Sep. 14, 2015 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. patent application Ser. No. 15/227,813 (filed on Aug. 3, 2016 and entitled Robotic Surgical Devices, System and Related Methods”) U.S. patent application Ser. No. 15/599,231 (filed on May 18, 2017 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. patent application Ser. No. 15/691,087 (filed on Aug. 30, 2017 and entitled “Robotic Device with Compact Joint Design and an Additional Degree of Freedom and Related Systems and Methods”), U.S. patent application Ser. No. 15/821,169 (filed on Nov. 22, 2017 and entitled “Gross Positioning Device and Related Systems and Methods”), U.S. patent application Ser. No. 15/826,166 (filed on Nov. 29, 2017 and entitled “User controller with user presence detection and related systems and methods”), U.S. patent application Ser. No. 15/842,230 (filed on Dec. 14, 2017 and entitled “Releasable Attachment Device for Coupling to Medical Devices and Related Systems and Methods”), U.S. patent application Ser. No. 16/144,807 (filed on Sep. 27, 2018 and entitled “Robotic Surgical Devices with Tracking Camera Technology and Related Systems and Methods”), U.S. patent application Ser. No. 16/241,263 (filed on Jan. 7, 2019 and entitled “Single-Manipulator Robotic Device With Compact Joint Design and Related Systems and Methods”), U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

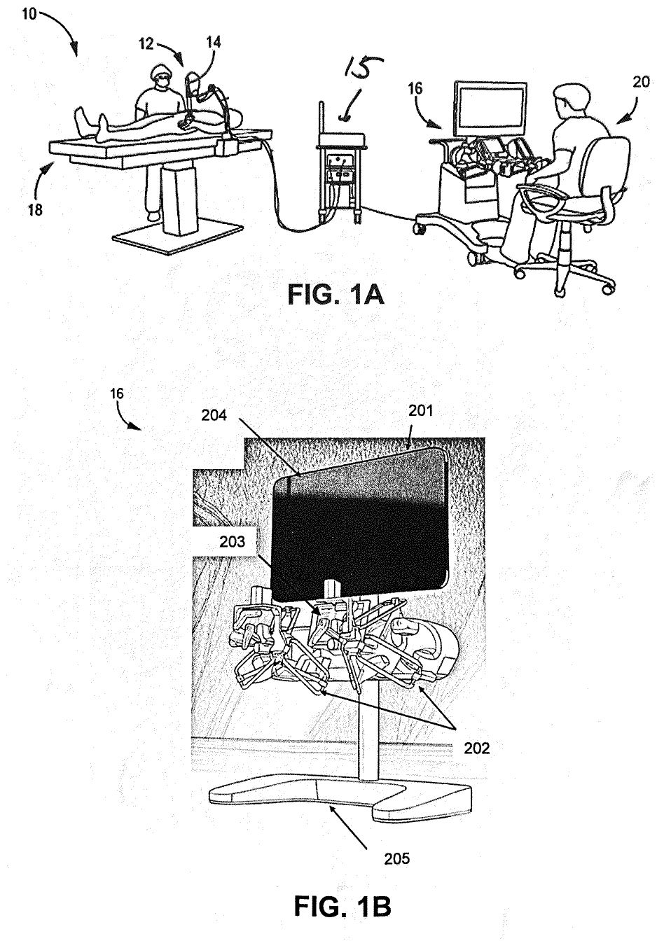

FIG. 1A depicts one embodiment of a robotic surgical system 10 having several components that will be described in additional detail below. The components of the various system implementations disclosed or contemplated herein can include an external control console 16 and a robotic device 12 having a removable camera 14 as will also be described in additional detail below. In accordance with the implementation of FIG. 1A, the robotic device 12 is shown mounted to the operating table 18 and coupled to the console 16 via a surgical cart (or “companion cart”) 15. The system 10 can be, in certain implementations, operated by the surgeon 20 at the console 16 and one surgical assistant positioned at the operating table 18. Alternatively, one surgeon 20 can operate the entire system 10. In a further alternative, three or more people can be involved in the operation of the system 10. It is further understood that the surgeon (or user) 20 can be located at a remote location in relation to the operating table 18 such that the surgeon 20 can be in a different city or country or on a different continent from the patient on the operating table 18.

One specific embodiment of an external control console 16 for use in a system (such as system 10) is depicted in addition detail in FIG. 1B. Here, the console 16 has a surgical display 201 and hand controllers 202. In this implementation, a secondary display and/or touchscreen 203 exists between the hand controllers 202. In some embodiments, the console 16 can also include foot pedals 205 in addition to or as an alternative to the hand controllers 202.

In certain implementations, the console 16 as shown in FIG. 1B can also have an integrated audio/visual system 204 including a camera (not shown) facing the surgeon (such as surgeon 20), a speaker (not shown), and a microphone (not shown) allowing the surgeon's voice to be recorded/transmitted and allowing the surgeon to hear others through the speaker. This integrated AV system with 2-way visual and audio communication enables remote communication that is useful in remote and/or tele-surgical applications where the surgeon 20 and console 16 might be remote to the surgical space and thus the patient. That is, the various system embodiments herein allow for the surgeon 20 and console 16 to be located anywhere in relation to the surgical space. In other words, the surgeon 20 and console 16 can be in the same room, a neighboring room, even in a different state, country or continent in relation to the surgical space. In such configurations, the integrated AV system 204 allows for communication between the surgeon 20 (and/or others at or near the console 16) and technicians or other people at other locations, including the surgical space (as will be described in additional detail below in relation to certain cart embodiments).

Various embodiments of the surgical devices as contemplated herein (such as device 12 as discussed above and other device embodiments as discussed in detail below) can also incorporate one or more 3D or stereo cameras, as will be discussed in additional detail below. Thus, certain versions of the console 16 that can be used in conjunction with such cameras are configured to allow for viewing of the 3D or stereo images created by such cameras. In one specific example, the primary surgical display 201 could be configured to display a three-dimensional (3D) stereoscopic image or any other type of image generated by a 3D or stereo camera.

Alternatively, any console 16 embodiment herein can have any known type of display that allows for the viewing of 3D or stereo images generated by such cameras. For example, the display can be a stereoscopic, autostereoscopic, holographic, and/or volumetric display. In some cases, such displays necessitate the use of glasses or headsets. For example, stereoscopic displays require glasses/headsets (like active shutter or polarized) to direct different images to each eye. In contrast, autostereoscopic displays are glasses-free and use technologies such as parallax barriers or lenticular lenses. On the other hand, with respect to holographic displays (which create true 3D images by combining multi-angle stereopsis and accurate depth cues), some such displays require glasses or headsets, while others don't. Volumetric displays (which can generate imagery within a genuine 3D space) typically do not require glasses or headsets.

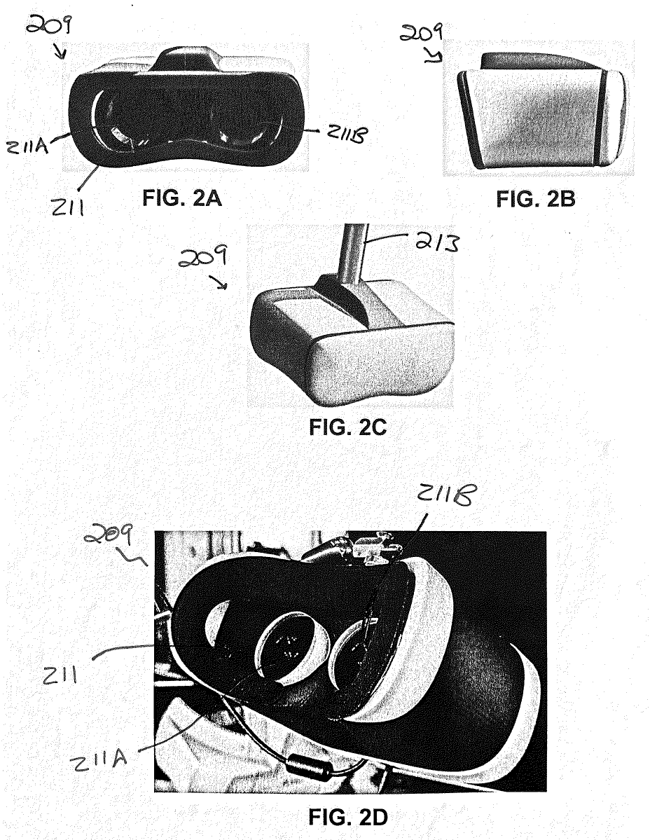

One non-limiting, exemplary implementation of a 3D display is shown in FIGS. 2A-2D, which depict an exemplary autostereoscopic 3D headset or display 209. As best shown in FIGS. 2A and 2D, the headset 209 has a viewfinder 211 through which a user can view the images generated by the headset 209. In addition, the viewfinder 211 in certain embodiments can have two lenses 211A, 211B. Further, the headset 209 can be attached to a related component (such as, for example, a console 16) via an attachment rod 213, as best shown in FIG. 2C, or in another fashion. Alternatively, the display 209 can be a true headset 209 that can be attached to the surgeon's (or another user's) head. In use, according to some embodiments, the autostereoscopic 3D headset 209 can serve as the primary surgical display 201 (replacing the flatscreen display 201 as shown in FIG. 1B) and thus be incorporated into the console 16 as best shown in FIG. 2D. In one specific example, the headset 209 can be a Quest 3 headset from Meta. Alternatively, the headset 209 can be any known VR or other type of headset 209 for use in such a system (such as system 10) to display the images of a 3D or stereo camera incorporated into the surgical device (such as device 12). The primary surgical display 201 and/or the headset 209 could be part of a console 16 such as either of the consoles illustrated in FIGS. 1A and 1B.

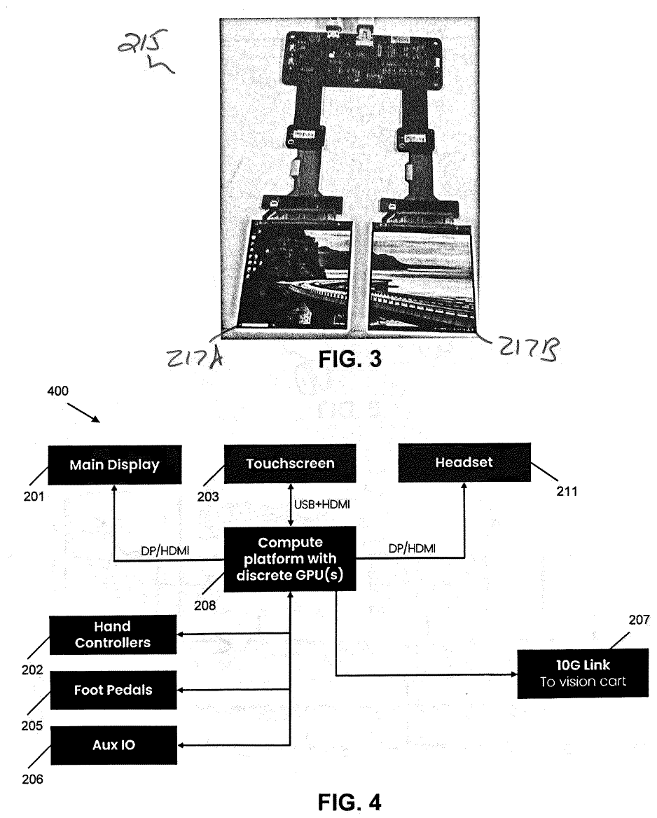

Various types of headsets and other 3D or stereo image displays are configured to operate by displaying two separate images as shown in an exemplary fashion in FIG. 3. More specifically, as shown via the representative screen setup 215, two separate screens 217A, 217B are provided, wherein each screen 217A, 217B depicts a different image. More specifically, in those embodiments in which the device camera is a stereo or 3D camera that captures two separate images, certain of the various 3D or stereo image displays (such as the headset 209, for example) have two screens 217A, 217B, with each screen 217A, 217B displaying a different one of the two separate images captured by the camera. (The specific setup 215 as shown uses two 1440×1440 resolution OLED displays 217A, 217B that are configured to display the left and right images.) As with any of the various displays that have two separate displays, the two separate displays 217A, 217B of this configuration 215 are effectively treated as one 2880×1440 display such that no frame syncing needs to be done. Thus, this configuration 215 and any of the 3D or stereo image headsets or displays as disclosed or contemplated herein can provide a 1:1 pixel mapping of the 3D camera as described in additional detail below.

In some embodiments such as that depicted in FIGS. 2A-2D, the two lenses 211A, 211B in the autostereoscopic 3D display (or any other display or headset embodiment contemplated herein) can be 50-millimeter diameter achromat doublets 211A, 211B with anti-reflective optical coatings that provide a high-contrast, ghost-free image, with minimal chromatic aberration. These optical properties allow for interpupillary adjustments to be completely done via software. Such a display 209 can be mounted to the console 216 as shown in FIG. 2D or head-mounted (as are many augmented reality commercial forms of this design). Any headset or display embodiment herein can, in certain implementations, replace the main display or could be used in conjunction with the main display. While display 209 has a mount or attachment rod 213 disposed above the display 209 as discussed above, other versions can have a mount or attachment structure disposed below the display 209 to attach it to the console 16, such as the version depicted in FIG. 2D.

In those console 16 embodiments that include a 3D or stereo display, the hardware configuration can be configured as shown in FIG. 4, which depicts a schematic diagram of the console hardware configuration 400, according to an embodiment. In this implementation, the hardware configuration 400 has a computer platform 208 that is operably coupled to all of the other components of the console 16. That is, the computer platform 208 is a known computer platform (or “compute platform”) 208 that operates as a computing system for use in operation of the console 16. In some exemplary embodiments, the compute platform 208 can contain a discrete Graphics Processing Unit (GPU). Alternatively, the compute platform 208 can have any processor, processing unit, and/or software that can be used for graphic processing. In one non-limiting example, the compute platform 208 is a NVIDIA IGX Orin, which is a commercially-available, industrial-grade, edge AI platform designed for use in demanding environments like medical and manufacturing settings. In some embodiments, as will be discussed further herein, the GPU/compute platform 208 can include an image signal processor (ISP) or can be configured to run a software based ISP.

Continuing with FIG. 4, the compute platform 208 is coupled to both the other console 16 components and to other system components, such as, for example, a surgical or companion cart/vision cart, such as companion cart 500, as will be discussed in additional detail below. That is, the compute platform 208 can be operably coupled to the one or more displays of the console 16 (such as a main display 201, a touchscreen 203, and/or a headset 211, for example), the controllers of the console 16 (such as hand controllers 202 and/or foot pedals 205), and any auxiliary input/output component 206) of the system 400. One of skill in the art understands that the compute platform 208 can be coupled to each of the components as discussed above via any known communication line or technology. For example, the compute platform 208 can be coupled to the controller(s), input/output components, and/or the surgery cart (such as cart 500) via an ethernet connection 207 (e.g. 10G or 10 Gigabit), serial, or other communication protocols. According to further examples, the compute platform 208 can be coupled with any display components (such as the main display 201, the touchscreen 203, and/or the headset 211 (or other autostereoscopic 3D display)) via HDMI or Display Port technology.

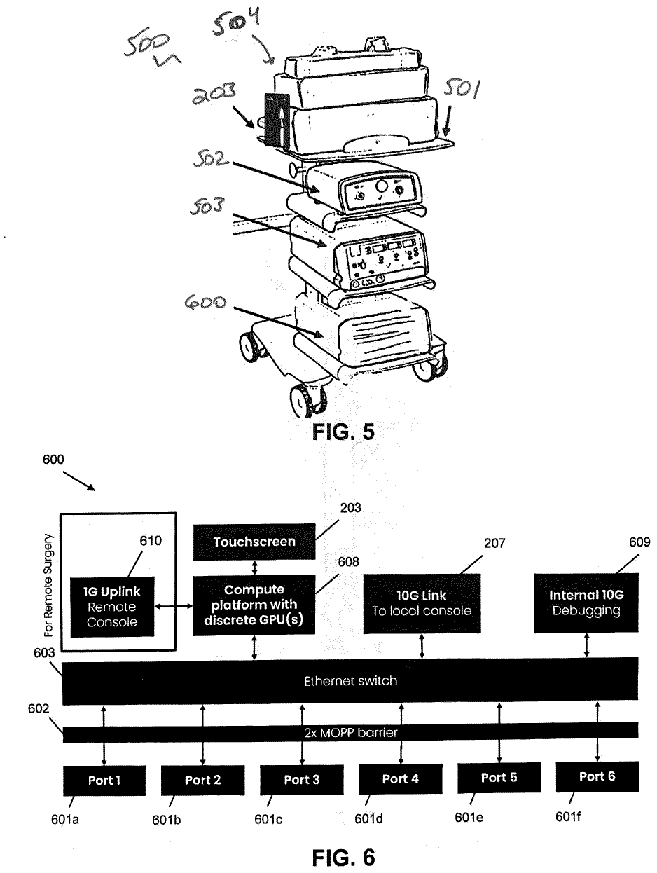

As noted above, in some embodiments, any surgical system herein (such as system 10 as discussed above) can also have a surgery or companion cart (such as cart 15 as discussed above with respect to FIG. 1A). According to another implementation, FIG. 5 depicts another version of a companion cart 500. This specific cart 500 has an interface 203 such as a touchscreen/display 203 mounted to the cart 500. This interface 203 enables those at the patient side (in the surgical space or arena) to interact with the system 10 and, in some cases, communication with and/or control functions of the console 16 (which may be at a different location). For example, the interface 203 could be used for things such as initiating remote surgery procedures, robot initialization, and adjusting ESU settings, among other things. In alternative embodiments, the interface 203 can be handheld, it could be wired or wireless, and it could be inside or outside the sterile field. This design of the cart 500 can be helpful for remote or tele-surgical applications where the control console 16 is remote from the surgical space and thus from the patient.

In this specific implementation, the companion cart 500 also has an integrated audio/visual system 504 including a camera facing the surgeon, a speaker, and a microphone allowing the surgeon's voice to be recorded/transmitted. In a similar fashion to the AV system 204 in the console 16 as discussed above, this AV system 504 enables remote communication that is useful in remote and/or tele-surgical applications where the surgeon and console 16 might be remote to the patient. The surgeon and console 16 could be in a neighboring room or at any other location across the country or world. That is, the AV system 504 of the surgical cart 500 allows for technicians and/or anyone else in the surgical space/near the patient to communicate via the system (such as system 10), including with the surgeon and/or anyone else at or near the console 16 (via the console AV system 204).

According to certain embodiments, this audio/visual equipment 504 can also be used to observe the technician and everything in the surgical environment by capturing video or other images and sound. Further, in additional implementations, this captured information can also be used for artificial intelligence inference and machine learning to determine parameters important to the surgery. In certain embodiments, the audio/visual equipment 204 on the cart 500 can be disposed on a mast or other similar structure and further might have pan/tilt/zoom or other moving capabilities that allow the AV system 504 to capture the best or desired images or sounds. In some embodiments, the companion cart 500 can also include a shelf or setup area 501. In an exemplary instance, the integrated audio/visual equipment 504 can be disposed on and/or within the setup area 501.

In certain versions, the cart 500 also has an electrosurgical energy unit/system 503 that can be used to operate the electrosurgical aspect of the robotic device (such as device 12). Thus, in one embodiment, the electrosurgical energy system 503 can have a vessel sealer mode that can be used to operate one or more vessel sealer end effectors on the device (such as device 12). Alternatively, an integrated electrosurgical energy system 503 can be integrated into the robot control module (aka connection pod) 502 or integrated into the cart 500 or elsewhere in the system (such as system 10) in another way.

Continuing with FIG. 5, the companion cart 500 can also have a control module 600. The control module 600 can operate to operate the surgical cart 500 and to communicate with the rest of the overall system (such as system 10), including with the console 16 and the compute platform 208.

A schematic of the companion cart control module 600 is shown in FIG. 6, according to one embodiment. In this exemplary embodiment, the control module 600 can have several ports 601a-f (referred to herein collectively as ports 601) configured to connect to robots, cameras, and other devices that are used during operation of the overall surgical system (such as system 10). The control module 600 can also have an electrical isolation barrier 602 that can be used to electrically isolate the various surgical devices coupled to the ports 601 from the various components of the module 600. More specifically, the electrical isolation barrier 602 in this embodiment is physically disposed between the ports 601 and the other components as shown. In one version, the barrier 602 is two means of patient protection (MOPP) 602. The electrical isolation of the barrier 602 can be accomplished through optical isolation or other forms of isolation. One of skill in the art understands that any known electrical isolation barrier 602 can be used.

In this specific implementation, the control module 600 can also have a an ethernet switch 603, which is a network device that connects multiple cabled devices, like computers and servers, within a Local Area Network (LAN). The switch 603 is used to connect any of the various devices attached at the ports 601 to any of the components of the module 600, the cart 500, or the overall system (such as system 10). Further, the control module 600 has a connection cable or link 207 by which a local console 16 can be coupled to the control module 600. More specifically, FIG. 4 illustrates the link 207 coupled to the compute platform 208 of the console 16, while FIG. 6 illustrates the link 207 coupled to the switch 603 of the control module 600 of the cart 500. Thus, in those embodiments in which the console 16 and surgical space are in the same location or in close proximity, the console 16 physically connects to the switch 603 via the link 207 for local surgery.

The control module 600 also has compute platform 608, which, in some embodiments, can be the same as or similar to the compute platform 208 of the console 16 as discussed above. In the control module 600, the compute platform 608 is configured to couple with the touchscreen 203 of the cart 500 and also with an uplink connection (or other remote communication component) 610 for remote surgery (1G, 10G, or 100G ethernet or other). That is, the remote communication component 610 can communicate wirelessly or via any known form of communication with a console 16 that is disposed at a different location in relation to the cart 500. In certain versions of the control module 600, such a remote communication component may not be needed when all surgical procedures are performed locally (such that the console 16 and the cart 500 are always in the same space or area or nearby. Alternatively, various implementations of this system (such as system 10) can have both the cable 207 and the remote communication component 610, thereby allowing for both a local console and a remote console to interact with the system at the same time. In some embodiments, the companion cart control module 600 can also have an internal debugging component 609 for internal debugging.

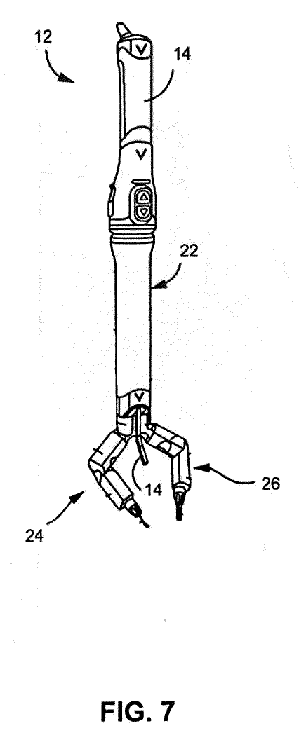

FIG. 7 depicts the robotic device 12, which can be incorporated into the exemplary system 10 discussed above or used with any other system disclosed or contemplated herein. The device 12 has a body (or “torso”) 22 with an imaging device (or “camera”) 14 disposed therethrough, as mentioned above and as will be described in additional detail below. Briefly, the robotic device 12 has two robotic arms 24, 26 operably coupled thereto and the camera 14 is removably positionable through the body 22 and disposed between the two arms 24, 26. That is, device 12 has a first (or “right”) arm 24 and a second (or “left) arm 26, both of which are operably coupled to the device 12 as shown and discussed in additional detail below. Each arm 24, 26 can have an upper arm and a forearm as shown or alternatively can have any known configuration. Further, in various embodiments, the arms are configured to receive various removeable, interchangeable end effectors.

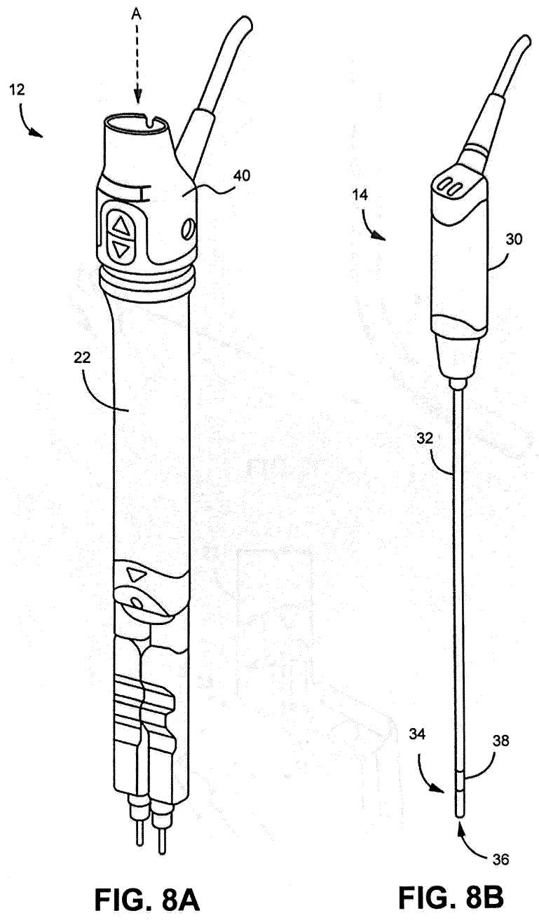

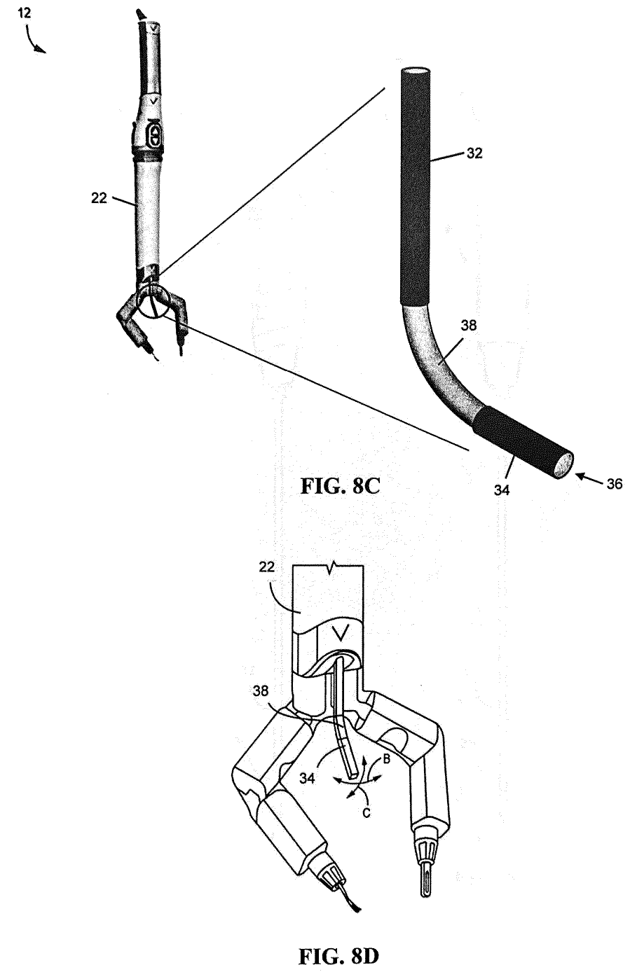

FIGS. 8A and 8B depict one embodiment of the robotic device 12 with the camera assembly 14 removed, according to one implementation. That is, FIG. 8A depicts the device 12 without the camera positioned through the body 22, and FIG. 8B depicts one embodiment of the camera 14. In certain implementations, and as best shown in FIG. 8B, the camera 14 has a handle (or “camera body”) 30 with an elongate shaft 32 coupled thereto such that the shaft 32 extends distally from the distal end of the body 30. In addition, the camera 14 has a steerable tip 34 coupled to the distal end of the shaft 32 via a flexible section 38 such that the steerability allows the user to adjust the viewing direction, as will be discussed in further detail below. Further, the tip 34 also includes a camera imager 36 at the distal end of the tip 34 that is configured to capture the desired images. Further, the tip 34 in certain implementations has an illumination light (not shown) disposed thereon, such that the light can illuminate the objects in the field of view. In one specific implementation, the camera 14 provides 1080 p 60 Hz. digital video. Alternatively, the camera 14 can provide any known video quality.

As best shown in FIGS. 8A, 8C, and 8D, the camera assembly 14 can be inserted into the body 22 of the robotic device 12 by positioning the distal end of the shaft 32 through a lumen (not shown) defined through the body 22 of the robotic device 12 as shown by the arrow A in FIG. 8A. As will be described in further detail below, certain implementations of the device 12 include a removable nest (or “dock”) 40 disposed near the proximal end of the body 22 that includes a seal (not shown) that operates to ensure that the patient's cavity remains insufflated. When the shaft 32 is inserted through the lumen of the body 22 as desired, according to certain embodiments as best shown in FIGS. 7, 8C, and 8D, the distal end of the shaft 32, including the flexible section 38 and the steerable tip 34 (containing the imager 36) extends out of an opening at the distal end of the body 22 such that the tip 34 is positioned between the two arms 24, 26 in the surgical environment as shown. Thus, the imager 36 is positioned to capture the view between the two arms 24, 26 and the steerable tip 34 can be actuated to provide views of the surgical tools and surgical target. That is, the tip 34 can be moved (such as, for example, in two different directions, including the yaw direction as represented by arrow B and the pitch direction as represented by arrow C) such that the surgical tools and/or surgical target are captured within the field of view of the imager 36. Alternatively, the tip 34 can move in any known way. It is understood that this camera 14 embodiment and any other such camera embodiment disclosed or contemplated herein can be used with any similar robotic device having a camera lumen defined therethrough. In various implementations, the camera 14 can be controlled via a console (such as console 16 discussed above, for example) or via control buttons (not shown) as will be discussed in additional detail below. In one embodiment, the features and operation (including articulation) of the steerable tip are substantially similar to the steerable tip as described in U.S. applications Ser. No. 14,334,383 and Ser. No. 15/227,813, both of which are incorporated by reference above. Alternatively, any known robotic articulation mechanism for cameras or similar apparatuses can be incorporated into any camera embodiment utilized in any device or system disclosed or contemplated herein.

In various implementations, the camera 14 can be re-sterilized for multiple uses. In one specific embodiment, the camera 14 can be reused up to one hundred times or more. Alternatively, it is understood that any known endoscopic camera that can fit through a device body according to any implementation herein can be utilized.

The various camera embodiments herein (including camera 14, for example) can, in certain implementations, be coordinated with the device to which it is coupled to create coordinated triangulation between the camera and the arms and end effectors for any configuration, positioning, and use of the device. Further, the steerable tip of any such camera can be robotically articulated so as to reposition the field of view, either automatically or via control by the surgeon using the system console. That is, the camera articulates to ensure the surgeon can view all possible locations of the robotic arms as well as the desired areas of the surgical theater. Further, as the robotic arms move-the steerable camera tip can be coordinated with the arms to move using active joints in coordination with the arm movements to view the entire robot workspace. In certain implementations, the joints of the camera are actively controlled using motors and sensors and a processor (and, in some implementations, a control algorithm contained therein). In these implementations, the processor allows for automated and/or semi-automated positioning and re-positioning of the camera 12 about the pitch (α) and/or yaw (β) rotations relative to the robotic device. It is understood that the various embodiments of systems and devices having such a coordination between the camera and the device (and arms) and the resulting features thereof are disclosed in detail in U.S. Published Application 2019/0090965, which is incorporated by reference above.

Alternatively, in certain implementations, the camera 14 can be removed from the robotic device 12 and positioned through another, known laparoscopic port typically used with a standard manual laparoscope. As such, in this embodiment, the device 12 is disposed through a main port (also known as an “insertion port”) and the camera 14 is positioned through the known laparoscopic port as shown. It is understood that this arrangement may be useful to visualize the robotic device 12 to ensure safe insertion and extraction via the main port. According to various embodiments, the camera 14 can also be removed from the robotic device 12 so the optics can be cleaned, the camera 14 can be repaired, or for any other reason in which it is beneficial to remove the camera 14. It is understood that while the device 12 and camera 14 are depicted and discussed herein, any device or camera according to any implementation disclosed or contemplated herein can also be used in a similar arrangement and any such camera can also be removed from the device for any reason as discussed herein.

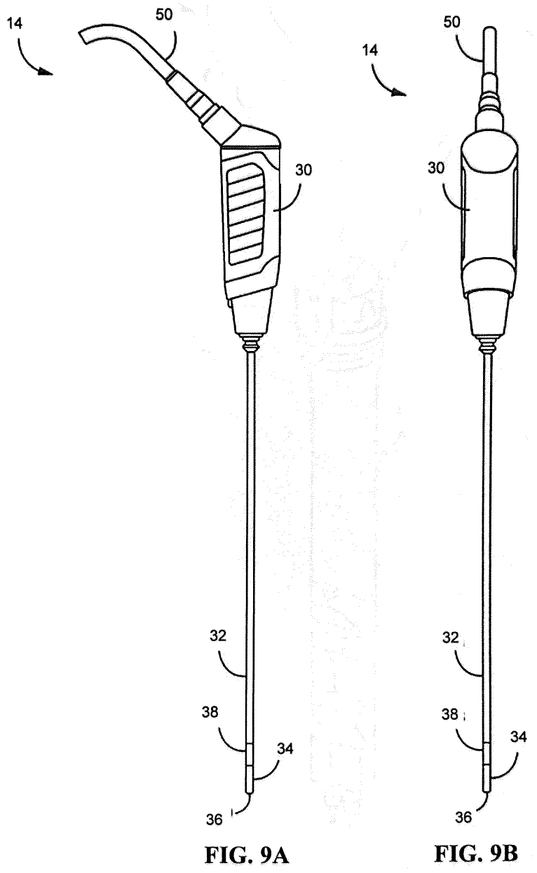

The camera (also referred to as a “camera assembly”) 14—according to one embodiment—is depicted in additional detail in FIGS. 9A and 9B (with FIG. 9A depicting a side view and FIG. 9B depicting a front view), in which the camera assembly 14 with the camera body 30 and elongate shaft 32 are shown. The shaft is coupled to the body 30 such that the shaft 32 extends distally from the distal end of the body 30. In addition, as also discussed above, the steerable tip 34 is coupled to the distal end of the shaft 32 via the flexible section 38, which couples the tip 34 to the shaft 32 such that the steerability allows the user to adjust the viewing direction, as discussed above and in further detail below. The tip 34 includes a camera imager (also referred to as an “imaging sensor”) 36 at the distal end of the tip 34 that is configured to capture the desired images, along with optics and support electronics (not shown), as will also be discussed in further detail below. The camera 14 can also have light fibers (not shown) that are disposed through the shaft 32, flexible section 38, and tip 34 such that the light fibers provide light output at the tip 34 so as to light the surgical target for imaging. In addition, the assembly 14 has a cable 50 that is coupled to the handle 30 and extends therefrom to an external controller (such as the console 16 discussed above or any other controller) such that the cable 50 can provide electrical signals to and from the camera 14, including a video signal and any power and other signals or information necessary to operate the camera 14.

It is noted that the camera body 30 in the various embodiments herein can have various components therein for operation of the camera assembly 14. For example, any camera body 30 herein can be substantially similar to the camera body implementations disclosed in U.S. Pat. No. 11,903,658, which is hereby incorporated herein by reference in its entirety. Alternatively, the camera body 30 can have any known components or features for movement of the steerable tip 34 and/or actuation or operation of the imaging sensor 36.

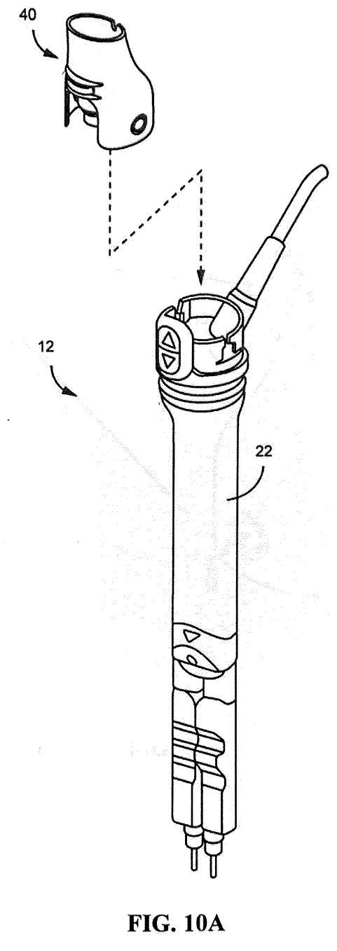

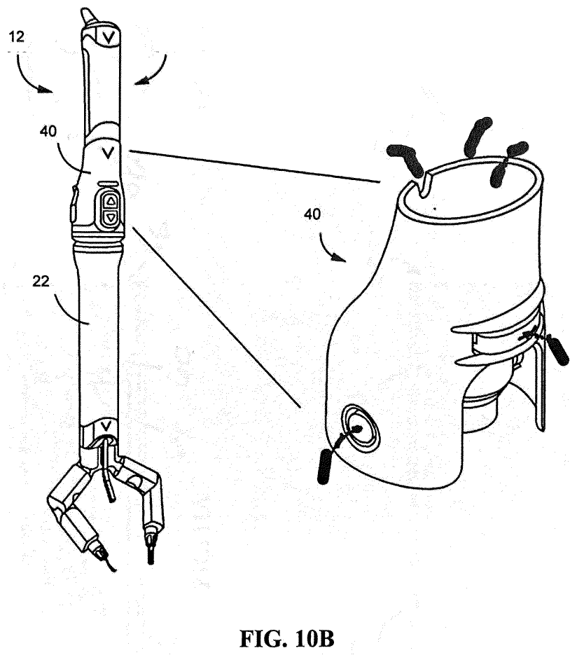

As noted above and shown in additional detail in FIGS. 10A and 10B, various implementations of the robotic device 12 can include a removable nest 40 that is removably coupleable to a proximal end of the device body 22 (as best shown in FIG. 10A) and is designed to receive the insertable camera assembly 14 (including, for example, any camera embodiment disclosed or contemplated herein) and couple or lock the device 12 and camera 14 together, as is shown in FIG. 10B. That is, the nest 40 is a coupling component or port that can couple to both the device 12 and camera 14 as shown such that the nest 40 is disposed between the device body 22 and the camera 14 when the three components 22, 14, 40 are coupled together (as best shown in FIG. 10B). In one exemplary embodiment, the nest 40 incorporated herein can be any nest embodiment as disclosed in U.S. Pat. No. 11,903,658, which is incorporated herein above.

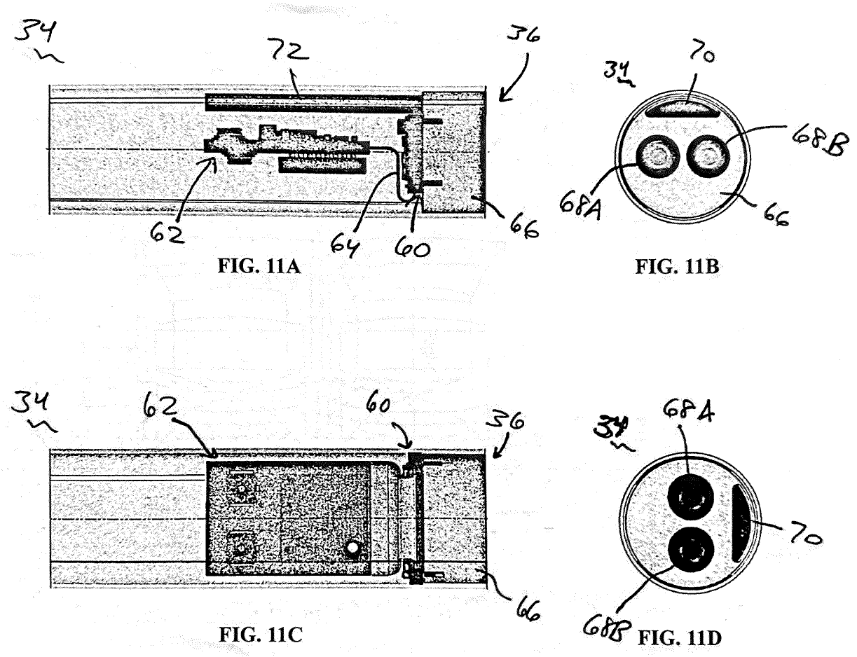

In one exemplary implementation, the tip 34 of the camera assembly 14 is depicted in FIGS. 11A-11D in additional detail such that the camera imager 36 and other internal components are shown. (One of skill in the art understands that the word “tip” as used herein is intended to describe the body (or “housing”) 34 at the distal end of the elongate shaft 32 of the camera assembly 14.) More specifically, FIGS. 11A and 11B depict the body 34 in one position, while FIGS. 11C and 11D depict the body 34 in another position. That is, FIG. 11A is a side view of the tip body 34 and FIG. 11B is a front view of the distal end of the tip 34, and in both figures, the tip 34 is positioned with the light output 70 disposed at the top of the tip 34. In contrast, FIG. 11C is a side view and FIG. 11D is a front view in which the tip 34 is positioned with the light output 70 disposed on the side of the tip 34. In other words, in FIGS. 10C and 10D, the tip 34 has been rotated 90 degrees (in comparison to FIGS. 11A and 11B) such that a different view of the internal components is provide in FIG. 11C. In FIGS. 11A-11C the light output 70 can be described as being disposed at or near an outer edge of the lens housing 66 and/or the tip 34 and further can be described as being disposed at or near an outer circumference of the lens housing 66.

With reference to FIGS. 11A-11D, according to one embodiment, the camera imager 36 in the tip body 34 can include the following components: an image sensor 60, communication and support components 62 operably coupled to the image sensor 60 via at least one electrical cable 64, a lens housing 66 disposed distally of the image sensor 60, two lenses 68A, 68B disposed within the housing 66, and a light output 70 disposed within the housing 66 that is operably coupled to light fibers 72 extending proximally from the output 70 as shown.

In this implementation, camera imaging assembly 36 has a single image sensor 60 as shown. The image sensor 60 can be a printed circuit board (“PCB”) 60 in certain embodiments. Alternatively, any known image sensor 60 for use in surgical cameras can be used.

According to certain embodiments, the communication and support components 62 can include any standard, commercially available components that come with or are necessary for the operation of the image sensor 60.

As noted above, the two lenses 68A, 68B are disposed distally of the image sensor 60 such that each lens 68A, 68B captures a different image that is directed through the lens and onto the sensor 60. Any known lens for use in surgical cameras can be used.

As discussed above, the light output (or “light”) 70 is disposed within the lens housing 66 at the distal end of the tip housing 34. More specifically, the light 70 is configured to emit light outward from the distal end of the tip body 34 to illuminate the area captured by the image sensor 60 via the lenses 68A, 68B. Further, the light fibers 72 operably coupled to the light output 70 are configured to direct light from an external source along the length of the fibers 72 to the output 70. While only a short length of the fibers 72 is shown, one of ordinary skill understands that the fibers 72 extend proximally along the entire length of the camera shaft 32, through the body 30 and out via the cable 50 to the external source. Any known light output 70 for use in surgical cameras can be used.

In this embodiment as shown in FIGS. 11A-11D, the single image sensor 60 makes it possible to reduce the size of the distal tip body 34 and thus the overall device 12. In contrast to a standard stereo camera having two image sensors mounted side by side as described above, the single image sensor 60 in this exemplary implementation allows for reduced overall dimensions.

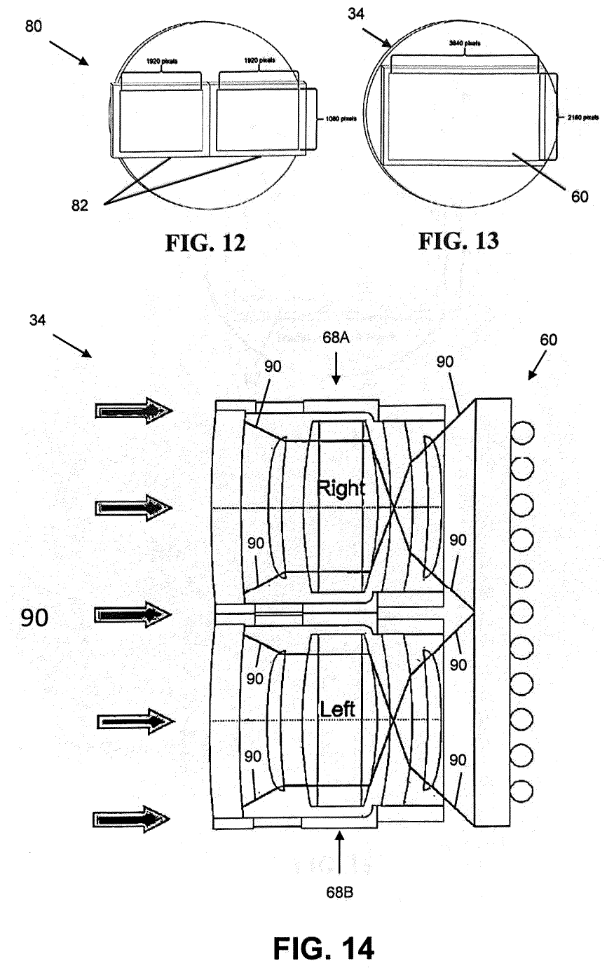

This can best be understood with reference to FIGS. 12 and 13. That is, FIG. 12 depicts a schematic representation of a standard stereo camera 80 with two side-by-side image sensors 82. In contrast, FIG. 13 depicts a cross-sectional image of the single image sensor 60 of the tip body 34.

The presence of two image sensors (such as sensors 82) instead of one sensor (such as sensor 60) creates space inefficiencies. This inefficiency stems from the additional support circuity (the communication and support components similar to those components 62 above) that is necessary for operation of each image sensor and that also provides additional functionality (such as, for example, PLL circuitry for clocks, voltage regulation/gating, image signal processor (ISP) circuitry, and high-speed I/O circuitry (MIPI transceivers, I2C peripherals, etc.). For example, the popular OmniVision OV2740, widely used in surgical laparoscopic imaging, has an active imaging area of 2.73 mm×1.55 mm, but is housed in an integrated circuit package that is 3.85 mm×2.89 mm, meaning that only 37.6% of the overall package area is used for imaging.

Thus, returning to the contrast between FIGS. 12 and 13, when packed in a circular tube (the industry standard form-factor for a multitude of reasons), the use of one image sensor 60 instead of two image sensors 82 can result in a 20% reduction in cross-sectional area, while yielding as many as twice as many pixels (in one example: 1920×1080×2=4.15 MP vs 3840×2160=8.29 MP).

Any of the camera components incorporated into any of the robotic devices as contemplated herein can have a dual lens-stack architecture. Such a configuration makes it possible to project left and right images onto a single CMOS image-sensor array. For example, FIG. 14 depicts a top down view of a tip housing 34 of a camera having a dual lens-stack architecture. More specifically, the housing 34 has two lenses 68A and 68B that are “stacked” or otherwise disposed adjacent to each other in the housing 34 as shown. In the exemplary instance as shown, lens 68A is the right lens and lens 68B is the left lens. The use of two lenses 68A, 68B results in the formation of image circles over the entire sensor area of image sensor 60, thereby allowing both images to be captured on a single device. This dual lens configuration reduces the circuit-board complexity required for image acquisition and ensures that the left and right images remain inherently synchronized. For instance, as illustrated via the light path depicted in FIG. 14, when a light input 90 travels through the stacked lenses 68A, 68B, the light 90 and resulting images are projected over the entire sensor area of the image sensor 60.

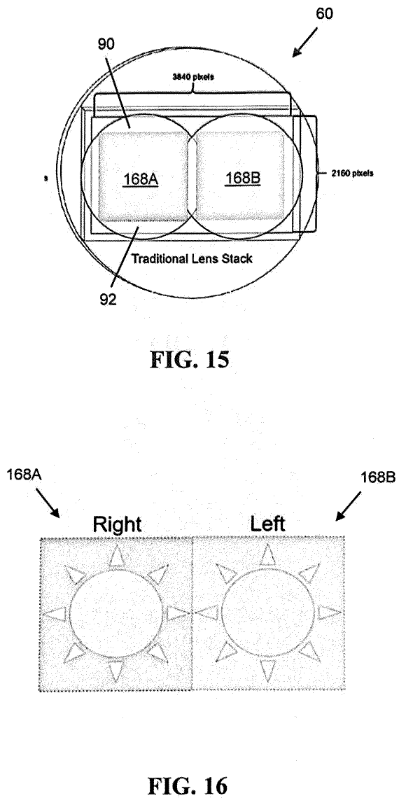

FIG. 15 depicts one example of a lens stack design for a single image sensor 60. As discussed above, using a single image sensor 60 can yield more pixels in contrast to using two image sensors 82. For example, FIG. 12 (which shows the use of two image sensors 82) illustrates an image having 1920×1080×2 pixels, whereas FIG. 15 illustrates an image having 3840×2160 pixels due to the use of a single image sensor 60. In other words, the single image sensor design of FIG. 15 results in more pixels than the two image sensor configuration of FIG. 12. As also shown in FIG. 15, in some embodiments, the use of stacked lenses 68A, 68B results in two images on the image sensor 60: a right image 168A and a left image 168B.

Despite resulting in more pixels, the use of a dual stacked lens design and a single image sensor still results in some unused pixels. For example, as shown in FIG. 15 in the standard display style (with no anamorphic lens design), the square image format (with right image 168A and left image 168B) results in sections of unused pixels at the top 90 and bottom 92 of the image sensor 60. However, as illustrated in FIG. 16, the resulting image with right image 168A and left image 168B is uniformly scaled and is not distorted. Thus, the resulting image does not need to be interpolated or otherwise modified to un-distort the image.

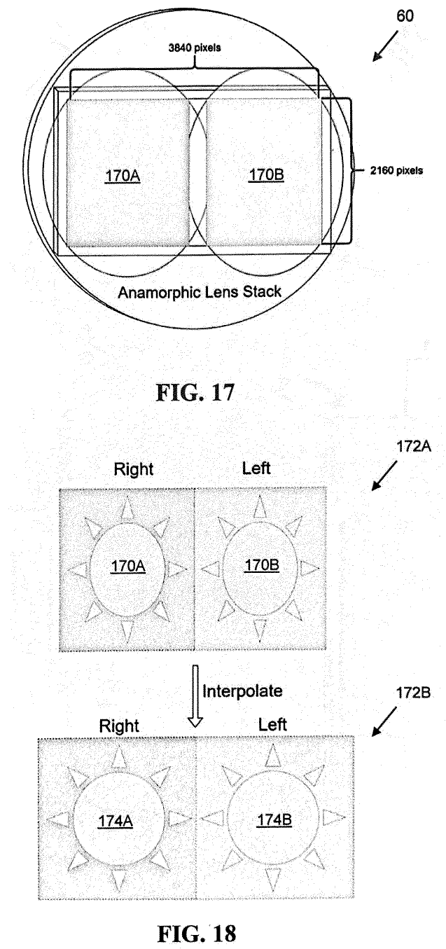

In certain implementations, an anamorphic lens design can be incorporated into any of the camera embodiments contemplated herein to maximize the number of pixels used. In contrast to the standard lens design discussed above in relation to FIGS. 15 and 16, an anamorphic lens design can be incorporated as depicted in FIG. 17 to maximize the number of pixels used in the image sensor by altering the image via the lens such that the image is stretched out to use the pixels in the image sensor 60 not used in the standard display style. This image (having right image 170A and left image 170B) is intentionally scaled non-uniformly along its vertical axis. This results in an elongated, distorted image 170A, 170B relative to the true appearance due to the anamorphic lens asymmetric elements. This vertical distortion allows for more of the available pixel area in the vertical direction to be used. For instance, the elongated, distorted image 170A, 170B for the anamorphic lens stack illustrated in FIG. 17 does not have any unused pixels (such as the unused pixels at the top 90 and bottom 92 of the image sensor 60 illustrated in FIG. 15). This increased sensor coverage of the anamorphic lens stack design can also enhance the effective field of view.

FIG. 18 illustrates the resulting image 172 before (172A) and after (172B) interpolation. For instance, image 172A illustrates the resulting image in its distorted state. The distorted image 172A has elongate distortion of the images 170A, 170B in the image sensor. The distorted image 172A can then be interpolated or otherwise modified to result in an undistorted image 172B. The undistorted image 172B includes modified right and left images 174A, 174B that are no longer elongated and are properly scaled. This correction or modification may be performed through pixel-mapping, warping, or interpolation algorithms that counteract the anisotropic magnification introduced by the anamorphic lens stack. Suitable interpolation techniques include, but are not limited to, bilinear interpolation, spline-based geometric transforms, Lanczos resampling, directional cubic convolution interpolation, and other similar methods.

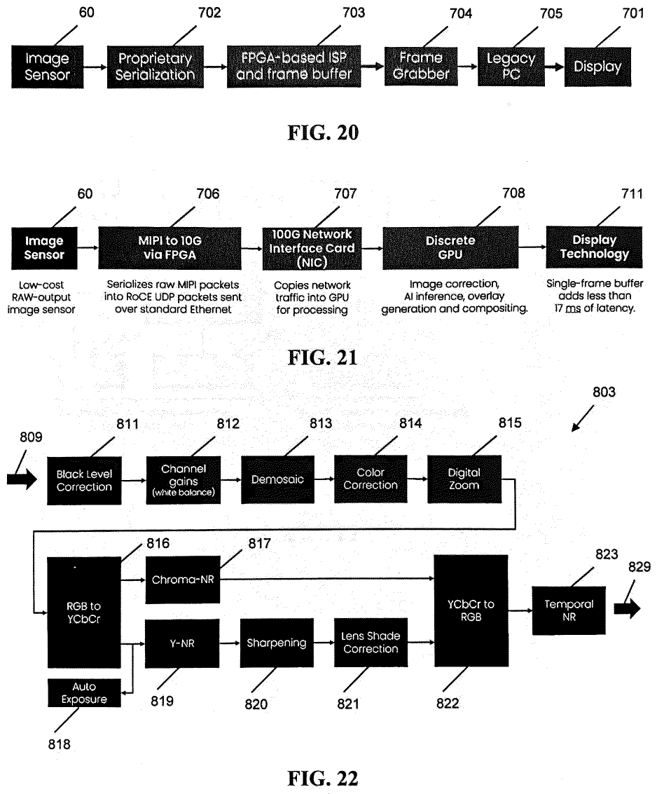

In those implementations in which an anamorphic lens is used, these correction operations can be integrated into a broader image-processing pipeline (for example, an image processing system (ISP)) that may also include black-level correction, lens-shading correction, white balance, demosaicing, color correction, luma and chroma noise reduction (temporal and spatial), cropping, auto-exposure adjustments, color balancing, and/or gamma correction. The combined processing ensures that the output image 172B exhibits restored geometric proportions as well as accurate color and luminance characteristics. For example, as discussed in additional detail below, these correction operations could be integrated into a pipeline such as the pipeline depicted in FIG. 20, the pipeline depicted in FIG. 21, and/or the image processing system depicted in FIG. 22.

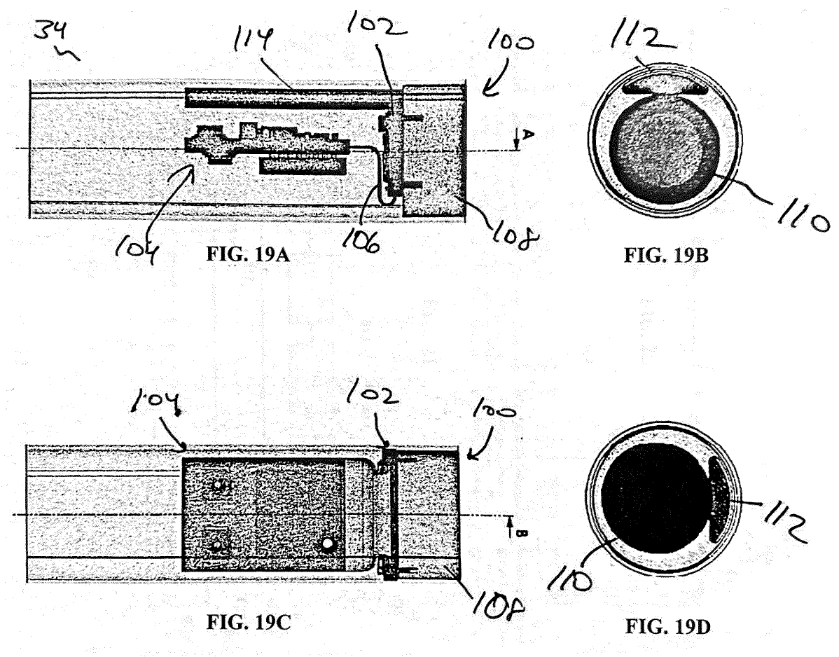

With reference to FIGS. 19A-19D, according to another camera embodiment, another camera imager 100 in the tip body 34 can include the following components: an image sensor 102, communication and support components 104 operably coupled to the image sensor 102 via at least one electrical cable 106, a lens housing 108 disposed distally of the image sensor 102, a single lens 110 disposed within the housing 108, and a light output 112 disposed within the housing 108 that is operably coupled to light fibers 114 extending proximally from the output 112 as shown.

In accordance with one embodiment, the camera imager 100 has components and features that are substantially similar to or identical to the corresponding components and features in the imager 36 discussed above, except as expressly described herein. Thus, as noted above, in this implementation, the camera imaging assembly 100 has a single lens 110 as shown.

FIG. 20 shows a traditional/standard video pipeline. In this context, one of skill in the art understands that “video pipeline” means the entire process of capturing and ultimately displaying an image in the context of a medical imaging system. Here, in the first step of the pipeline, an image sensor (such as single image sensor 60 discussed herein) captures the visual information (such as the target tissue captured by the device camera) and outputs it in one or more of various forms (e.g. MIPI-mobile industry processor interface). This is generally a high speed and high volume process, so a serial communication system such as a proprietary serial communication system 702 may be used to transmit the data out of the camera 14 in a proprietary serialization step. This information is received by a floating point gate array (FPGA), which is the hardware used in this process. At the FPGA, the image signal processing (ISP) 703 is used to modify the data (via such processes as demosaicing, noise reduction, white balance, exposure, lens correction, brightness, etc.). In some instances, the FPGA 703 is a FPGA-based ISP and frame buffer 703. This ISP 703 reconstructs a full-color image from the raw data captured by the sensor, which captures only one color per pixel. The reconstructed image is then transmitted and displayed on a display 701. In some embodiments, to transmit the reconstructed image from the ISP module 703 to the display 701, a frame grabber 704 can be used to grab and transmit the image to a computer 705 (such as a legacy PC, as an example). The computer can then display the image on display 701. In an exemplary embodiment, during use of the pipeline of FIG. 20 in any of the various system embodiments herein (such as system 10), the display 701 can be the same as or similar to one or more of the displays discussed herein (such as surgical display 201, display 211 (for example, with displays 217A, 217B and/or achromat doublets displaying right and left images 168A, 168B), etc.) and/or another type of display.

According to another embodiment, FIG. 21 shows an alternative video pipeline that is configured for use with any system herein (such as system 10) in which a stereo or 3D camera and corresponding stereo or 3D display are used. This alternative approach, which includes some of the same and/or corresponding steps to those in the standard pipeline of FIG. 20, involves the use of an FPGA-based interface board 706. The FPGA-based interface board 706 is an exemplary serial communication system that transmits visual information from the image sensor 60 to other component(s). For instance, in certain embodiments, this board 706 can encapsulate the sensor data into user datagram protocol (UDP) packets over ethernet and send it to the host system/software 707 (e.g., NVIDIA IGX or Jetson AGX Orin). For instance, the board 706 can serialize raw MIPI packets into RoCE UDP packets sent over standard ethernet (such as 10G Ethernet, as an example). The host software 707 receives this data via a high-speed network interface card (NIC) (for example, a 100G NIC) and makes it available to the processing pipeline. For example, the host software 707 can copy network traffic into GPU for processing. In this system, the host is a discrete GPU 708 that can run a software based Image Singal Processor (ISP). This GPU 708 can then also perform many functions like digital zoom, AI inference, etc. In some exemplary instances, the discrete GPU 708 can be the same as or similar to one or more GPUs discussed herein, such as GPU 208, compute platform 608, and/or another type of GPU.

The GPU 708 can then output the video for display on display technology 711. In an exemplary embodiment, the display 711 can be the same as or similar to one or more of the displays discussed herein (such as surgical display 201, display 211 (for example, with displays 217A, 217B and/or achromat doublets displaying right and left images 168A, 168B), etc.) and/or another type of display. Display technology 711 can be the same as or similar to display 701, in an exemplary embodiment.

Alternatively, any video pipeline for use in similar systems and/or with stereo/3D cameras and displays can be used with any of the overall system embodiments disclosed or contemplated herein (such as system 10). Further, either of the video pipelines described above can be performed with any optional steps removed and/or additional optional steps added.

In certain embodiments, any of the compute platforms (such as platforms 208, 608, 708) or GPUs disclosed or contemplated herein can include a software-based Image Signal Processor (ISP) system 803 as shown in FIG. 22. Thus, such a system 803 can operate on any such platform or GPU. Further, in various implementations, such a system 803 could additionally or alternatively be the same as or similar to ISP 703. In this embodiment, the ISP 803 operates in the following fashion. First, image data 809 is brought into the ISP 803. In an exemplary embodiment, the image data 809 could already be serialized via serialization such as serialization 702 or 706. In another exemplary embodiment, the image data 809 could be brought into the ISP 803 from host software 707.

Continuing with FIG. 22, the ISP system 803 can include black level correction 811 in accordance with some versions. This can adjust the darkest part of an image to ensure that pure black appears as a value close to zero. Black level correction 811 can be important for dynamic range. Further, the next step can involve white balance 812, also referred to as channel gains 812, which can adjust the colors in the image to ensure white objects appear white. In various implementations, the channel gains 812 can be fixed or automatic, depending on the light source. In some embodiments, the next step is demosaic 813, which reconstructs a full-color image from the raw data captured by the sensor, thereby capturing only one color per pixel. Next, a color correction step 814 can be performed, which adjusts colors for proper appearance. After that, a digital zoom 815 step can be performed, which crops and enlarges a section of an image in software. Further, another step—or steps—can relate to noise reduction. Noise reduction reduces or removes visual noise from the image. In ISP system 803, noise reduction can include at least chroma, luminance, and temporal. Thus, several different noise reduction steps (816, 817, 819, 822, 823) are interspersed throughout the process. For instance, each of the RGB (red, green, blue) to YCbCr (luminance (Y) and chrominance (Cb and Cr)) step 816, the Y-NR step 819, and the YCbCr to RGB step 822 can be luminance noise reduction. Further, Chroma-NR step 817 is chroma noise reduction, while the temporal NR step 823 is temporal noise reduction. Intermixed with those various noise reduction steps (816, 817, 819, 822, 823), can be the auto exposure 818, sharpening 820, and lens shade correction 821 steps. Auto exposure 818 automatically adjusts the brightness and exposure of the image. Sharpening 820 enhances edges within an image, typically done on the luminance via unsharp mask or other techniques like Laplacian, as an example. Lens shade correction 821 corrects for distortions and shading caused by the camera lens. In some embodiments, the ISP system 803 also includes tone mapping to adjust the overall brightness and contrast to make the image more visually appealing. Steps 811, 812, 813, 814, 815, 816, 817, 818, 819, 820, 821, 822, and 823 may be referred to herein as modules, and each module may be configured to perform their respective operation(s) discussed above. The resulting image and/or video 829 from the process 803 can be displayed (for example, through display 701, display technology 711, and/or any other display discussed herein) and/or transmitted to other components (such as the frame grabber 704, computer 705, and/or another component discussed herein).

Alternatively, any ISP process or system for use in similar overall systems and/or with stereo/3D cameras and displays can be used with any of the overall system embodiments disclosed or contemplated herein (such as system 10). Further, the ISP process 803 described above can be performed with any optional steps removed and/or additional optional steps added.

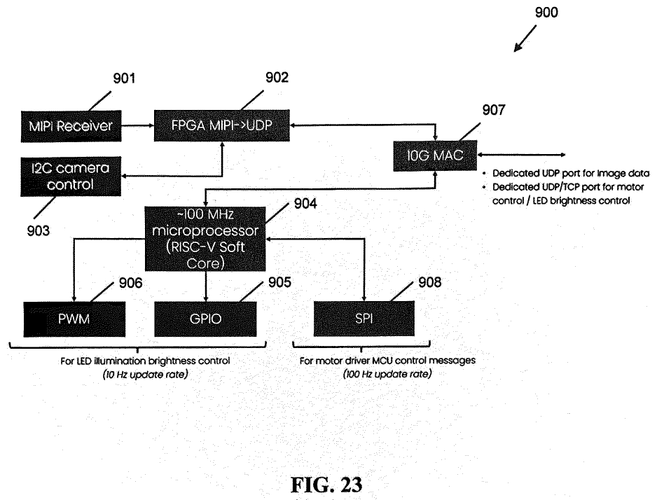

According to certain versions, more is involved with controlling the camera in the various systems disclosed or contemplated herein (such as system 10) than just the video pipeline (such as the pipelines depicted in FIGS. 20 and/or 21). That is, FIG. 23 shows a schematic of an overall system 900, according to one embodiment. In this exemplary system 900, a receiver 901, such as an MIPI receiver, is provided that can receive image data. Further, in this particular embodiment, the FPGA-based interface board 902 that encapsulates the image sensor data into user datagram protocol (UDP) packets over ethernet 907 (illustrated as 10G ethernet media access controller (MAC) 907) and sends it to the host system can also be used to communicate to the image sensor 60 (not depicted in FIG. 23) to control various aspects of its operation (e.g. exposure level). This can be accomplished over an I2C (inter-integrated circuit) communication bus/camera control 903. In some embodiments, the FPGA-based interface board 902 can be the same as or similar to FPGA/ISP 703 and/or FPGA-based interface board 706. Alternatively, the board 902 can be any known FPGA-based interface board. The host system can be a host system such as host system 707, in an exemplary embodiment. The ethernet connection 907 can also allow for communication with a microprocessor (e.g. RISC-V Soft Core) 904 that can then control other functions of the camera 14 including general purpose input/output (GPIO) 905, and/or pulse width modulation (PWM) 906 for control of the illumination system. It can also provide communication to another microprocessor via serial peripheral interface (SPI) 908 to control the motion of various motors that articulate the camera. Other functions can also be controlled via other communication protocols.

Alternatively, any overall system like system 900 can be used with any of the overall surgical system embodiments disclosed or contemplated herein (such as system 10). Further, the system 900 described above can be performed with any optional steps removed and/or additional optional steps added.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A camera assembly for a robotic surgical system, the camera assembly comprising:
(a) an elongate camera shaft;
(b) a camera body coupled to a proximal end of the elongate camera shaft; and
(c) a steerable tip disposed at a distal end of the elongate camera shaft, the steerable tip comprising:
(i) a steerable tip body comprising:
(A) a single image sensor;
(B) one or more lenses disposed distally of the single image sensor; and
(C) an illumination component; and
(ii) a flexible section coupled to the elongate camera shaft and the steerable tip body,
wherein the steerable tip body is movable in relation to the elongate camera shaft.

2. The camera assembly of claim 1, wherein the one or more lenses comprise a first lens and a second lens, and wherein the first lens captures a first image and the second lens captures a second image.

3. The camera assembly of claim 2, wherein the first lens and the second lens form image circles over an entire sensor area of the single image sensor such that the first image and the second image are configured to be captured on the single image sensor.

4. The camera assembly of claim 2, wherein the first lens and the second lens have a traditional lens design or an anamorphic lens design.

5. The camera assembly of claim 1, wherein the illumination component is positioned towards an outer edge of a lens housing of the steerable tip body.

6. The camera assembly of claim 1, wherein the steerable tip body further comprises:
(a) communication and support components operably coupled to the single image sensor; and
(b) a lens housing disposed distally of the single image sensor;
wherein the illumination component is disposed within the lens housing and is operably coupled to light fibers extending proximally from the illumination component.

7. The camera assembly of claim 1, further comprising a communication system configured to transmit visual information from the single image sensor out of the camera assembly, wherein the visual information comprises one or more images captured by the one or more lenses.

8. The camera assembly of claim 7, wherein the communication system is a serial communication system.

9. A camera system for a robotic surgical system, the camera system comprising:
(a) a camera assembly, the camera assembly comprising:
(i) an elongate camera shaft;
(ii) a camera body coupled to a proximal end of the elongate camera shaft; and
(iii) a steerable tip disposed at a distal end of the elongate camera shaft, the steerable tip comprising:
(A) a steerable tip body comprising a single image sensor, an illumination component, and one or more lenses, the one or more lenses disposed distally of the single image sensor; and
(B) a flexible section coupled to the elongate camera shaft and the steerable tip body;
(b) an image signal processor (ISP) operably coupled to the single image sensor; and
(c) a surgical display operably coupled to the ISP, wherein the surgical display is configured to display one or more images captured by the one or more lenses.

10. The camera system of claim 9, further comprising a communication system configured to transmit visual information from the single image sensor to the ISP, wherein the visual information comprises one or more images captured by the one or more lenses.

11. The camera system of claim 10, wherein the one or more lenses have a traditional lens design or an anamorphic lens design.

12. The camera system of claim 11, wherein
the one or more lenses have an anamorphic lens design,
the one or more images are distorted one or more images,
the communication system is configured to transmit the distorted one or more images to the ISP, and
the ISP is configured to correct the distorted one or more images.

13. The camera system of claim 9, wherein the ISP comprises:
(i) a black level correction module;
(ii) a white balance module;
(iii) a Demosaic module;
(iv) a color correction module;
(v) a digital zoom module;
(vi) one or more noise reduction modules;
(vii) an auto exposure module;
(viii) a sharpening module; and
(ix) a lens shade correction module.

14. The camera system of claim 9, further comprising a display console, wherein the display console comprises the ISP and the surgical display.

15. The camera system of claim 14, wherein the display console comprises a discrete GPU and wherein the discrete GPU is configured to run the ISP.

16. A robotic surgical system, comprising:
(a) a robotic surgical device comprising:
(i) an elongate device body comprising a distal end and a proximal end;
(ii) a removable connection port disposed at the proximal end of the device body, the connection port comprising:
(A) a device body coupling mechanism disposed within the connection port;
(B) a camera receiving opening defined in a proximal end of the connection port; and
(C) a camera coupling mechanism disposed within the removable connection port; and
(iii) first and second robotic arms operably coupled to the distal end of the device body; and
(b) a removable camera component removably disposable in the camera receiving opening and through the seal package, the removable camera component comprising an elongate camera shaft, a camera body, a flexible section, and a steerable tip having a steerable tip body, the steerable tip body comprising:
(i) a single image sensor;
(ii) one or more image lenses disposed distally of the single image sensor; and
(iii) an illumination component; and
(c) a control console, the control console comprising a surgical display configured to display one or more images captured by the one or more lenses.

17. The robotic surgical system of claim 16, wherein the surgical display comprises at least one of a single screen, a plurality of screens, and a pair of achromat doublets.

18. The robotic surgical system of claim 16, wherein:
the one or more lenses comprise a first lens and a second lens, and
the surgical display comprises a first display and a second display, the first display corresponding to the first lens and the second display corresponding to the second lens.

19. The robotic surgical system of claim 16, wherein the control console further comprises:
(a) hand controllers; and
(b) an audio/visual system.

20. The robotic surgical system of claim 16, wherein the control console further comprises a graphics processing unit (GPU) operatively connected to the single image sensor, the GPU configured to run an image signal processor (ISP) to process visual information from the single image sensor and construct the one or more images to display on the surgical display.
